(19) United States
(12) Patent Application Publication
(10) Pub. No.: US 2017/0319261 A1
(43) Pub. Date: Nov. 9, 2017

(54) MEDICAL TREATMENT DEVICE

(71) Applicant: OLYMPUS CORPORATION, Tokyo (JP)

(72) Inventor: Hikaru INOUE, Tokyo (JP)

(73) Assignee: OLYMPUS CORPORATION, Tokyo (JP)

(21) Appl. No.: 15/661,364

(22) Filed: Jul. 27, 2017

Related U.S. Application Data
(63) Continuation of application No. PCT/JP2015/054031, filed on Feb. 13, 2015.

(51) Int. Cl. A61B 18/12; A61B 18/14; A61B 18/00

(52) U.S. Cl. CPC A61B 18/1206; A61B 18/1442; A61B 2018/00767; A61B 2018/00702; A61B 2018/00184; A61B 2018/128; A61B 2018/0072

(57) ABSTRACT

A medical treatment device includes a pair of holding members configured to grasp a target part to be connected and dissected in a body tissue. At least one holding member of the pair of holding members includes an energy application portion having a treatment surface configured to contact the target part when the target part is grasped by the pair of holding members to apply energy to the target part. The treatment surface includes a high-output area configured to apply energy to the target part at a first output value for dissecting at least the target part, and a low-output area configured to apply energy to the target part at a second output value smaller than the first output value. The high-output area and the low-output area are provided continuously to each other.

### CROSS REFERENCES TO RELATED APPLICATIONS

This application is a continuation of PCT international application Ser. No. PCT/JP2015/054031, filed on Feb. 13, 2015 which designates the United States, incorporated herein by reference.

### BACKGROUND

### 1. Technical Field

The disclosure relates to a medical treatment device.

### 2. Related Art

Conventionally, there has been known medical treatment devices for applying energy to body tissues to perform a treatment (connection (or anastomose) and dissection) on the body tissues (for example, see WO 2003/057058, FIG. 16).

WO 2003/057058 discloses a medical treatment device that includes a pair of jaws for grasping a part to be treated (hereinafter, referred to as a target part) in a body tissue, and also includes a sealing device and a dissecting device which are provided at one jaw of the pair of jaws.

The sealing device has a shape extending in a linear shape and contacts the target part when the target part is grasped by the pair of jaws. The sealing device applies high-frequency energy to the target part in accordance with high-frequency power which is supplied across the sealing device and an electrode provided at the other jaw so that the target part is in a close contact state.

The dissecting device has a linear shape and is disposed in parallel to the sealing device. When the target part is grasped by the pair of jaws, the dissecting device contacts the target part. The dissecting device applies heat energy to the target part to dissect the target part.

In the medical treatment device disclosed in WO 2003/057058, the sealing device and the dissecting device are separated from each other. For this reason, the following situation arises.

FIGS. 14A and 14B are diagrams illustrating a situation arises by the conventional medical treatment device. Specifically, FIGS. 14A and 14B are diagrams illustrating the target part TP having been treated by the medical treatment device disclosed in WO 2003/057058. In FIGS. 14A and 14B, a part to which energy is applied is indicated by hatching.

When the sealing device and the dissecting device are separated from each other, as illustrated in FIG. 14A, the treated target part TP includes a part PL which is in a close contact state due to the high-frequency energy applied from the sealing device, a part PH which is dissected due to the heat energy applied from the dissecting device, and a part PN which is not connected due to no energy applied thereto and is provided between the parts PL and PH.

Since the part PH is cauterized by the application of the heat energy, the part PH has a relatively strong connection strength. However, since the part PH is a necrotic part, the part PH is easily separated from the target part TP as illustrated in FIG. 14B. This separation is particularly likely to occur when there is the part PN in a non-connected state.

In this way, when the part PH is separated from the target part TP, the part PN is bifurcated as illustrated in FIG. 14B since the part PN is in a non-connected state. When the part PN is in a bifurcated state, the part PL is not completely connected due to the tissue regeneration power. Therefore, the part PL is bifurcated like the part PN as time goes by.

That is, according to the medical treatment device disclosed in WO 2003/057058, the target part TP may be opened after treatment.

### SUMMARY

In some embodiments, a medical treatment device includes a pair of holding members configured to grasp a target part to be connected and dissected in a body tissue. At least one holding member of the pair of holding members includes an energy application portion having a treatment surface configured to contact the target part when the target part is grasped by the pair of holding members to apply energy to the target part. The treatment surface includes: a high-output area configured to apply energy to the target part at a first output value for dissecting at least the target part; and a low-output area configured to apply energy to the target part at a second output value smaller than the first output value. The high-output area and the low-output area are provided continuously to each other.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating a medical treatment system according to a first embodiment of the invention;

FIG. 2 is an enlarged view illustrating a front end part of a medical treatment device illustrated in FIG. 1;

FIG. 3 is an exploded perspective view illustrating a configuration of a first energy application portion illustrated in FIG. 2;

FIG. 4 is a block diagram illustrating configurations of a control device and a foot switch illustrated in FIG. 1;

FIG. 5 is a flowchart illustrating a connecting and dissecting process using the control device illustrated in FIG. 1 or 4;

FIG. 6A is a diagram illustrating a positional relation of first and second holding members and a pressing member during the connecting and dissecting process illustrated in FIG. 5 when viewed from a front end side of a grasping portion;

FIG. 6B is a diagram illustrating a positional relation of the first and second holding members and the pressing member during the connecting and dissecting process illustrated in FIG. 5 when viewed from the front end side of the grasping portion;

FIG. 6C is a diagram illustrating a positional relation of the first and second holding members and the pressing member during the connecting and dissecting process illustrated in FIG. 5 when viewed from the front end side of the grasping portion;

FIG. 7 is a diagram illustrating a change in impedance calculated in and after step S3 illustrated in FIG. 5;

FIG. 8 is a diagram illustrating a target part subjected to the connecting and dissecting process illustrated in FIG. 5;

FIG. 9A is a diagram illustrating an effect of the first embodiment of the invention;

FIG. 9B is a diagram illustrating an effect of the first embodiment of the invention;

FIG. 10 is a block diagram illustrating a configuration of a control device according to a second embodiment of the invention;

FIG. 11 is a flowchart illustrating a connecting and dissecting process using the control device illustrated in FIG. 10;

FIG. 12 is an enlarged view illustrating a front end part of a medical treatment device according to a third embodiment of the invention;

FIG. 13A is a diagram illustrating a positional relation of first and second holding members during a connecting and dissecting process according to the third embodiment of the invention when viewed from a front end side of a grasping portion;

FIG. 13B is a diagram illustrating a positional relation of the first and second holding members during the connecting and dissecting process according to the third embodiment of the invention when viewed from the front end side of the grasping portion;

FIG. 13C is a diagram illustrating a positional relation of the first and second holding members during the connecting and dissecting process according to the third embodiment of the invention when viewed from the front end of the grasping portion;

FIG. 14A is a diagram illustrating a situation of a conventional medical treatment device; and

FIG. 14B is a diagram illustrating a situation of the conventional medical treatment device.

### DETAILED DESCRIPTION

Exemplary embodiments of the present invention will be described with reference to the drawings. The present invention is not limited by the embodiments described below. The same reference signs are used to designate the same elements throughout the drawings.

### First Embodiment

Schematic Configuration of Medical Treatment System

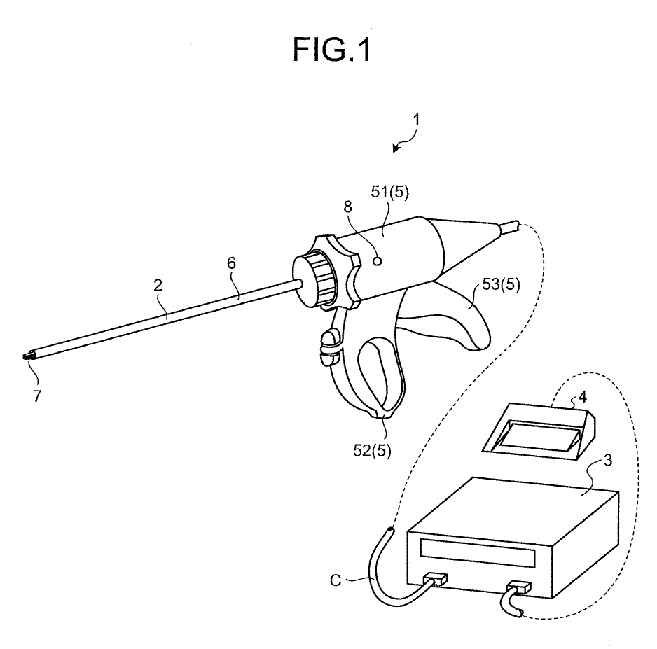

FIG. 1 is a schematic diagram illustrating a medical treatment system 1 according to a first embodiment of the invention.

The medical treatment system 1 applies energy (high-frequency energy and heat energy) to a part to be treated (connected (or anastomosed) and dissected) in a body tissue (hereinafter, referred to as a target part) to perform the treatment on the target part. As illustrated in FIG. 1, the medical treatment system 1 includes a medical treatment device 2, a control device 3 (overall controller), and a foot switch 4.

Configuration of Medical Treatment Device

The medical treatment device 2 is, for example, a linear type surgical medical treatment instrument for performing a treatment on the target part through an abdominal wall. As illustrated in FIG. 1, the medical treatment device 2 includes an operating unit 5 (operating device), a shaft 6, a grasping portion 7, and a display unit 8.

The operating unit 5 is a portion used by an operator to operate the medical treatment device 2. As illustrated in FIG. 1, the operating unit 5 includes a cylindrical portion 51 which has a cylindrical shape, a grip portion 52 which is integrally formed with the cylindrical portion 51 and is gripped by the operator, and an opening and closing operating unit 53.

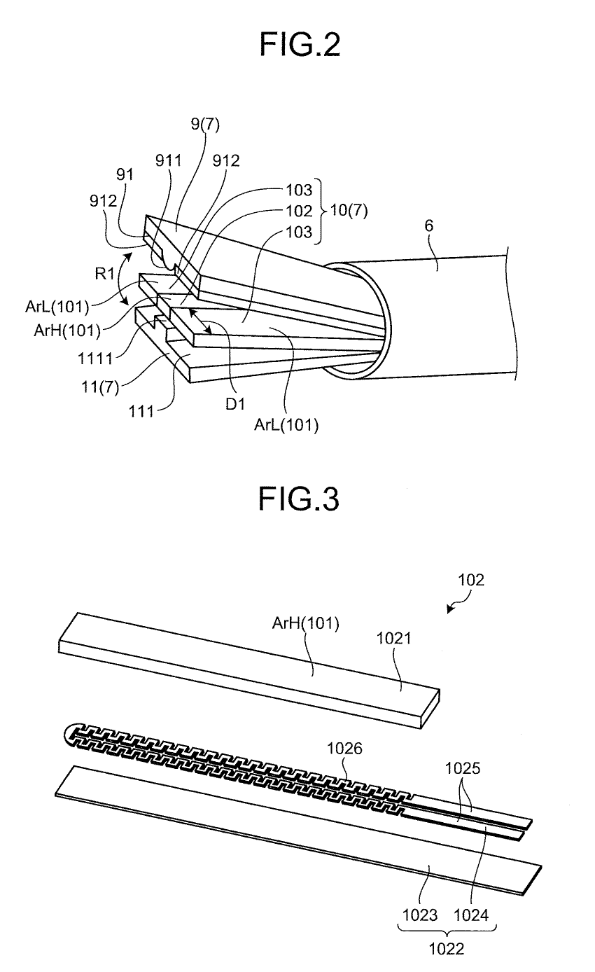

The opening and closing operating unit 53 is a portion operated by the operator when the grasping portion 7 is caused to perform a first opening and closing operation (an operation of opening and closing first and second holding members 9 and 10 (see FIG. 2)) and a second opening and closing operation (an operation of opening and closing a pressing member 11 (see FIG. 2) relative to the first and second holding members 9 and 10).

More specifically, the opening and closing operating unit 53 is supported by the cylindrical portion 51 to be movable along the axial direction of the cylindrical portion 51. The opening and closing operating unit 53 is connected to an opening and closing mechanism (not illustrated) provided inside the shaft 6 and allows the grasping portion 7 to perform the first opening and closing operation through the opening and closing mechanism in response to an operation (hereinafter, referred to as a first opening and closing operation) in which the opening and closing operating unit is moved by a predetermined amount in the axial direction (in FIG. 1, the left direction) of the cylindrical portion 51 by the operator. The opening and closing operating unit 53 allows the grasping portion 7 to perform the second opening and closing operation through the opening and closing mechanism in response to an operation (hereinafter, referred to as a second opening and closing operation) in which the opening and closing operating unit is moved further in the axial direction (in FIG. 1, the left direction) of the cylindrical portion 51 by the operator.

As illustrated in FIG. 1, the shaft 6 has a substantially cylindrical shape and one end thereof is connected to the operating unit 5 (the cylindrical portion 51). The grasping portion 7 is attached to the other end of the shaft 6. An electric cable C (FIG. 1) connected to the control device 3 is disposed inside the shaft 6 from one end side toward the other end side via the operating unit 5 in addition to the above-described opening and closing mechanism.

The display unit 8 is configured as, for example, an LED (Light Emitting Diode) or the like and is disposed to be exposed to the outer surface of the cylindrical portion 51 as illustrated in FIG. 1. The display unit 8 is electrically connected to the control device 3 via the electric cable C and is turned on under the control of the control device 3 so as to prompt the operator to perform the second opening and closing operation.

Configuration of Grasping Portion

FIG. 2 is an enlarged view illustrating a front end part of the medical treatment device 2.

The grasping portion 7 is a portion which performs a treatment on the target part by grasping the target part. As illustrated in FIG. 2, the grasping portion 7 includes the first holding member 9, the second holding member 10, and the pressing member 11.

The first and second holding members 9 and 10 and the pressing member 11 are axially supported by the other end of the shaft 6 to be openable and closable in the direction of the arrow R1 (FIG. 2) and are opened and closed in response to the first and second opening and closing operations by the operator.

The first holding member 9 is a portion which grasps the target part between the first holding member 9 and the second holding member 10. The first holding member 9 is formed as an elongated plate body extending in the axial direction of the shaft 6 and is disposed at the upper side of FIG. 2 relative to the second holding member 10 and the pressing member 11.

In the first holding member 9 illustrated in FIG. 2, a center portion of a lower plate surface 91 in the width direction is provided with a first convex portion 911 which protrudes downward and extends along the longitudinal direction of the plate surface 91 as illustrated in FIG. 2. On both sides of the first convex portion 911, a pair of electrodes 912 is disposed on the plate surface 91. The pair of electrodes 912 is electrically connected to the control device 3 via the electric cable C, and high-frequency power is supplied to the pair of electrodes 912 under the control of the control device 3.

Here, a cross-sectional shape of an outer surface of the first convex portion 911 is a substantially circular-arc shape. The most protruding position in the first convex portion 911 is located at the lower side in FIG. 2 relative to the pair of electrodes 912.

As illustrated in FIG. 2, the second holding member 10 is formed as a substantially elongated plate body similarly to the first holding member 9 and is disposed between the first holding member 9 and the pressing member 11. The second holding member 10 is a portion which grasps the target part between the first holding member 9 and the second holding member 10. In FIG. 2, an upper plate surface is formed as a treatment surface 101 contacting the target part.

The second holding member 10 serves as an energy application portion according to the invention and includes a first energy application portion 102 and a pair of second energy application portions 103 as illustrated in FIG. 2.

FIG. 3 is an exploded perspective view illustrating a configuration of the first energy application portion 102.

As illustrated in FIG. 2, the first energy application portion 102 is disposed at a position (a center portion of the second holding member 10 in the width direction) facing the first convex portion 911 in the first holding member 9 and applies heat energy to the target part from a high-output area ArH forming a part of the treatment surface 101. As illustrated in FIG. 3, the first energy application portion 102 includes a heat transfer plate 1021 and a heat generation sheet 1022.

The heat transfer plate 1021 is, for example, an elongated plate body formed of a material such as copper. The heat transfer plate 1021 transfers heat from the heat generation sheet 1022 to the target part (applies heat energy to the target part) while the high-output area ArH which is one plate surface contacts the target part.

The heat generation sheet 1022 serves as a sheet heater which partially generates heat and heats the heat transfer plate 1021 by the heat. As illustrated in FIG. 3, the heat generation sheet 1022 includes a substrate 1023 and a wiring pattern 1024.

The substrate 1023 is an elongated sheet which is formed of an insulating material such as polyimide.

The wiring pattern 1024 is formed by processing a metallic film formed on one surface of the substrate 1023 by lamination or vapor deposition and is used to heat the heat transfer plate 1021. As illustrated in FIG. 3, the wiring pattern 1024 includes a pair of lead wire connection portions 1025 and an electric resistance pattern 1026.

Here, a material of the wiring pattern 1024 is stainless or platinum.

The pair of lead wire connection portions 1025 extends from one end side (in FIG. 3, a right end side) of the substrate 1023 toward the other end side (in FIG. 3, a left end side) and are provided to face each other in the width direction of the substrate 1023. Two lead wires (not illustrated) constituting the electric cable C are respectively connected to the pair of lead wire connection portions 1025.

The electric resistance pattern 1026 has a U-shape along the outer edge of the substrate 1023 from one end of the pattern. One end of the pattern is connected (electrically connected) to one lead wire connection portion 1025, and the other end of the pattern is connected (electrically connected) to the other lead wire connection portion 1025. The electric resistance pattern 1026 generates heat by applying a voltage (electrically connecting) to the pair of lead wire connection portions 1025 via two lead wires by the control device 3.

The heat transfer plate 1021 is attached to a portion provided with the electric resistance pattern 1026 in the heat generation sheet 1022. Although not illustrated in the drawings in detail, an adhesive sheet is disposed between the heat transfer plate 1021 and the heat generation sheet 1022 to bond the heat transfer plate 1021 to the heat generation sheet 1022. This adhesive sheet is a sheet which has high thermal conductivity and adhesive properties while withstanding a high temperature. For example, the adhesive sheet is formed by mixing a ceramic having high thermal conductivity such as alumina or aluminum nitride with an epoxy resin.

As illustrated in FIG. 2, the pair of second energy application portions 103 is disposed on both sides of the first energy application portion 102, and respectively faces the pair of electrodes 912 of the first holding member 9. The pair of second energy application portions 103 applies high-frequency energy from each of the low-output areas ArL forming a part of the treatment surface 101 to the target part.

More specifically, the pair of second energy application portions 103 is, for example, an elongated plate body formed of a material such as copper and is electrically connected to the control device 3 via the electric cable C. The pair of second energy application portions 103 applies high-frequency energy to the target part when high-frequency power is supplied from the control device 3 to the pair of electrodes 912 while each low-output area ArL corresponding to one plate surface contacts the target part.

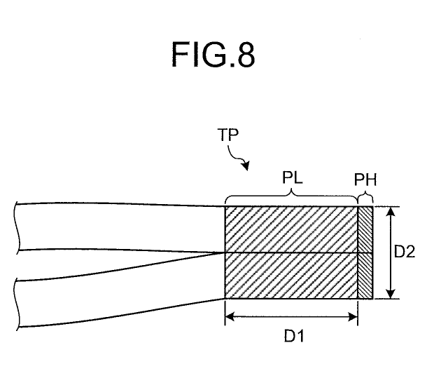

Here, each width D1 (FIG. 2) of the pair of second energy application portions 103 is set to be equal to or larger than a thickness D2 of the target part (see FIG. 8).

The above-described first and second energy application portions 102 and 103 are formed to be movable relative to each other.

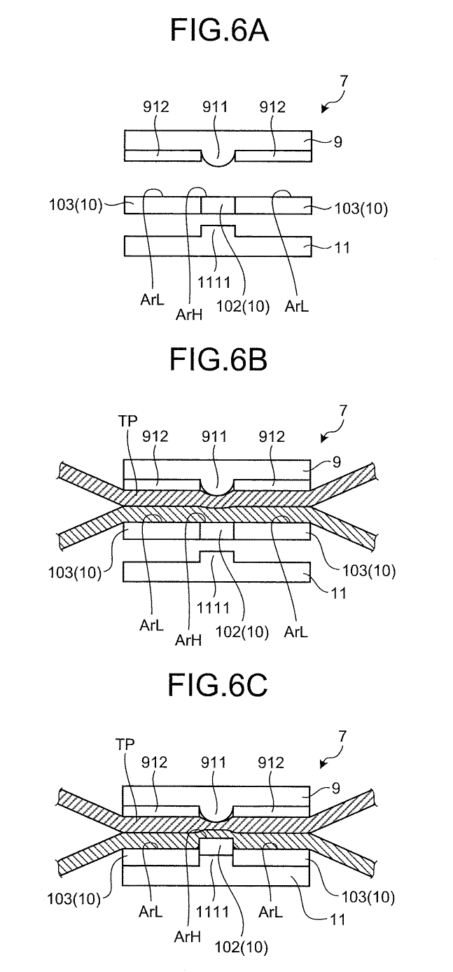

More specifically, the first and second energy application portions 102 and 103 are opened and closed relative to the first holding member 9 while keeping a first positional relation in which the high-output area ArH and each low-output area ArL are located at the same height position (the same level) in response to the first opening and closing operation (see FIGS. 6A and 6B). When the first energy application portion 102 is pressed toward the first holding member 9 at the pressing member 11 in response to the second opening and closing operation, the first and second energy application portions 102 and 103 are set to a second positional relation (a position in which the low-output areas ArL are located at the same height position) in which the high-output area ArH protrudes relative to each of the low-output areas ArL (see FIG. 6C).

That is, the high-output area ArH and the low-output areas ArL are provided continuously to each other in the width direction of the second holding member 10 while the first and second energy application portions 102 and 103 are set to the first positional relation. In other words, the high-output area ArH and the low-output areas ArL form a continuous surface which is the treatment surface 101 while the first and second energy application portions 102 and 103 are set to the first positional relation.

As illustrated in FIG. 2, the pressing member 11 is a portion which presses the first energy application portion 102 toward the first holding member 9. The pressing member 11 is formed as an elongated plate member similarly to the first holding member 9 and is disposed below the first and second holding members 9 and 10.

As illustrated in FIG. 2, a second convex portion 1111 which protrudes upward and extends in the longitudinal direction of a plate surface 111 is provided at a position (a center portion of the pressing member 11 in the width direction) facing the first energy application portion 102 in the upper plate surface 111 of the pressing member 11 in FIG. 2.

The second convex portion 1111 has a width which is the same as or slightly smaller than the width of the first energy application portion 102 and a face facing the first energy application portion 102 is formed in a flat shape. When the pressing member 11 moves in response to the second opening and closing operation, the second convex portion 1111 enters between the pair of second energy application portions 103 while pressing the first energy application portion 102 toward the first holding member 9.

Configuration of Control Device and Foot Switch

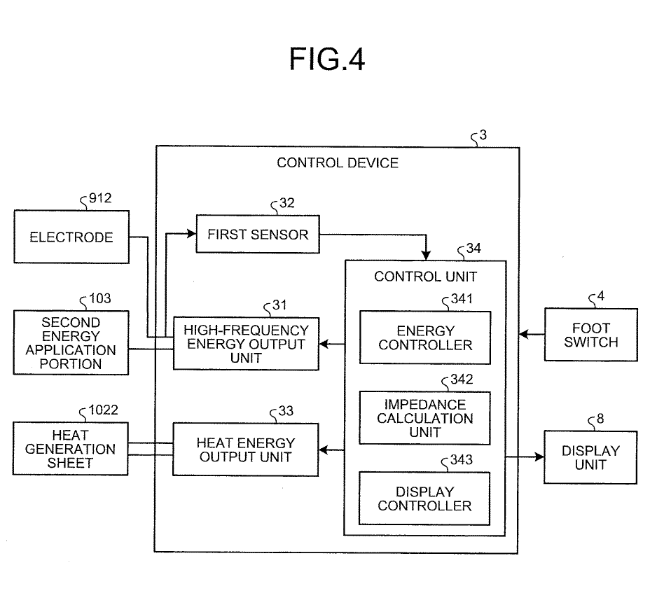

FIG. 4 is a block diagram illustrating configurations of the control device 3 and the foot switch 4.

Additionally, FIG. 4 mainly illustrates a main part of the invention as elements of the control device 3.

The foot switch 4 is a portion which is operated by a foot of the operator. The control device 3 starts a connecting and dissecting process to be described later in response to an operation (ON) for the foot switch 4.

Additionally, a unit that starts the connecting and dissecting process is not limited to the foot switch 4 and may be a switch that is operated by a hand.

The control device 3 generally controls the operation of the medical treatment device 2. As illustrated in FIG. 4, the control device 3 includes a high-frequency energy output unit 31, a first sensor 32, a heat energy output unit 33, and a control unit 34.

The high-frequency energy output unit 31 supplies high-frequency power to the pair of electrodes 912 and the pair of second energy application portions 103 via the electric cable C under the control of the control unit 34.

The first sensor 32 detects the values of a voltage and a current supplied from the high-frequency energy output unit 31 to the pair of electrodes 912 and the pair of second energy application portions 103. The first sensor 32 outputs a signal corresponding to the detected voltage and current values to the control unit 34.

The heat energy output unit 33 applies (energizes) a voltage to the heat generation sheet 1022 (the wiring pattern 1024) via the electric cable C under the control of the control unit 34.

The control unit 34 includes a CPU (Central Processing Unit) and the like and performs the connecting and dissecting process according to a predetermined control program when the foot switch 4 is turned on. As illustrated in FIG. 4, the control unit 34 includes an energy controller 341, an impedance calculation unit 342, and a display controller 343.

The energy controller 341 controls the operation of the high-frequency energy output unit 31 and the heat energy output unit 33 to control the output values of the heat energy and the high-frequency energy applied to the target part.

The impedance calculation unit 342 calculates an impedance (an impedance of the target part) when the high-frequency energy is applied to the target part based on the voltage and current values detected by the first sensor 32.

The display controller 343 prompts the operator to perform the second opening and closing operation by turning on the display unit 8 after the impedance calculated by the impedance calculation unit 342 reaches a minimum value. That is, the display unit 8 and the display controller 343 serve as an output device according to the invention.

Operation of Medical Treatment System

Next, an operation of the above-described medical treatment system 1 will be described.

In the following description, the connecting and dissecting process using the control device 3 will be mainly described as the operation of the medical treatment system 1.

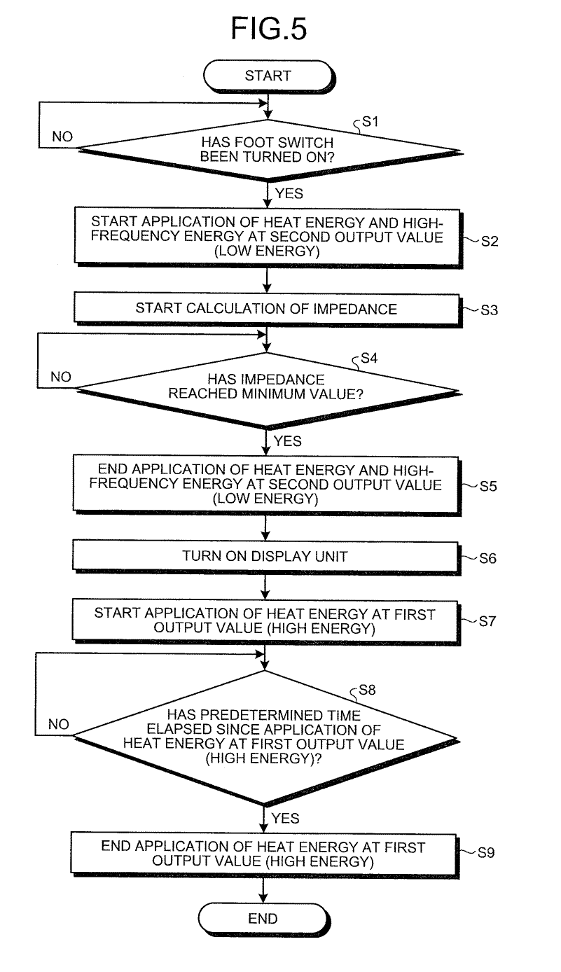

FIG. 5 is a flowchart illustrating the connecting and dissecting process using the control device 3. FIGS. 6A to 6C are diagrams illustrating a positional relation of the first and second holding members 9 and 10 and the pressing member 11 during the connecting and dissecting process when viewed from the front end side of the grasping portion 7. In addition, FIG. 6A illustrates a state where the first and second opening and closing operations are not performed by the operator. FIG. 6B illustrates a state where the first opening and closing operation is performed by the operator. FIG. 6C illustrates a state where the second opening and closing operation is performed by the operator.

The operator grips the medical treatment device 2 and inserts the front end part (a part of the grasping portion 7 and the shaft 6) of the medical treatment device 2 into an abdominal cavity through an abdominal wall using, for example, a troca or the like. Then, the operator performs the first opening and closing operation. In response to the first opening and closing operation, the first and second holding members 9 and 10 move close to each other to grasp the target part TP while the first and second energy application portions 102 and 103 keep the first positional relation as illustrated in FIGS. 6A and 6B.

Then, the operator operates (turns on) the foot switch 4 from the state illustrated in FIG. 6B to start the connecting and dissecting process using the control device 3.

When the foot switch 4 is turned on (step S1: Yes), the energy controller 341 starts the driving of the high-frequency energy output unit 31 and the heat energy output unit 33 so that the high-frequency energy and the heat energy are applied at the second output value from the first and second energy application portions 102 and 103 to the target part TP (step S2).

Here, the second output value is an output value for extracting at least an extracellular matrix of the target part TP. For example, an output value in which the target part TP becomes 80° C. or less is desirable.

That is, the type of energy applied to the target part TP is different. However, since the high-frequency energy and the heat energy are applied at the same second output value to the target part TP, the entire part grasped by the first and second holding members 9 and 10 at the target part TP has the substantially same temperature.

After step S2, the impedance calculation unit 342 starts the calculation of the impedance of the target part TP based on the voltage and current values detected by the first sensor 32 (step S3).

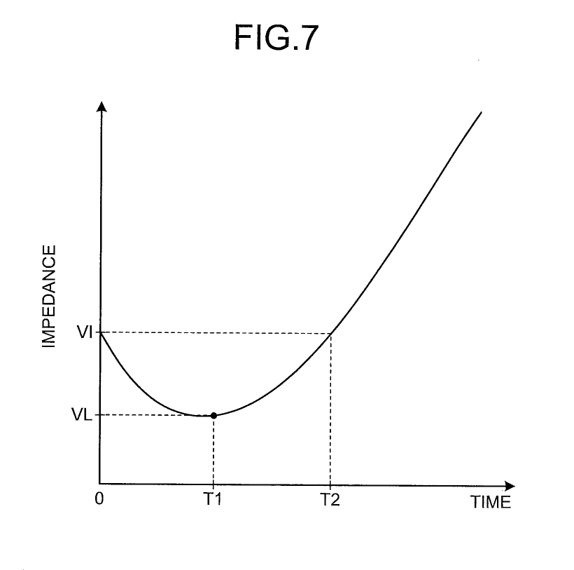

FIG. 7 is a diagram illustrating a change in impedance calculated in and after step S3.

When the high-frequency energy and the heat energy are applied to the target part TP at the second output value, the impedance of the target part TP changes as illustrated in FIG. 7.

At the initial time zone (the energy application start time T1) in which the high-frequency energy and the heat energy are applied at the second output value, the impedance gradually decreases as illustrated in FIG. 7. This is caused when the extracellular matrix is extracted from the target part TP due to the breakage of the cell membrane of the target part TP in accordance with the application of the high-frequency energy and the heat energy. In other words, at the initial time zone, the extracellular matrix is extracted from the target part TP and the viscosity of the target part TP decreases (the target part TP is softened).

From the time T1 at which the impedance reaches a minimum value VL, the impedance gradually increases as illustrated in FIG. 7. This is caused when the moisture inside the target part TP decreases (evaporates). In other words, after the time T1, the extracellular matrix is not extracted from the target part TP and the moisture inside the target part TP evaporates due to heat so that the viscosity of the target part TP increases (i.e., the target part TP coagulates).

After step S3, the control unit 34 normally monitors whether the impedance calculated by the impedance calculation unit 342 reaches the minimum value VL (step S4).

When it is determined that the impedance reaches the minimum value VL (step S4: Yes), the energy controller 341 stops the driving of the high-frequency energy output unit 31 and the heat energy output unit 33 (ends the application of the high-frequency energy and the heat energy to the target part TP at the second output value) (step S5).

After step S5, the display controller 343 turns on the display unit 8 so as to prompt the operator to perform the second opening and closing operation (step S6).

Then, the operator performs the second opening and closing operation by recognizing whether the display unit 8 is turned on. As illustrated in FIGS. 6B and 6C, the pressing member 11 (the second convex portion 1111) presses the first energy application portion 102 toward the first holding member 9 in response to the second opening and closing operation so that the second positional relation of the first and second energy application portions 102 and 103 is set. That is, in the target part TP, a part which is grasped by the first energy application portion 102 and the first convex portion 911 is grasped at a pressure higher than those of the other parts.

After step S6, the energy controller 341 starts the driving of the heat energy output unit 33 so that the heat energy is applied at the first output value from the first energy application portion 102 to the target part TP (step S7).

Here, the first output value is an output value for dissecting the target part TP. For example, an output value in which the target part TP becomes 200° C. or more is desirable.

That is, since the heat energy is applied at the first output value from the first energy application portion 102 to the target part TP, a part which is grasped by the first energy application portion 102 and the first convex portion 911 at the target part TP becomes a temperature of 200° C. or more.

After step S7, the energy controller 341 normally monitors whether a predetermined time elapses after the heat energy is applied at the first output value in step S7 (step S8).

Then, when it is determined that the predetermined time elapses (step S8: Yes), the energy controller 341 stops the driving of the heat energy output unit 33 (ends the application of the heat energy at the first output value to the target part TP) (step S9).

FIG. 8 is a diagram illustrating the target part TP subjected to the connecting and dissecting process. In FIG. 8, a part to which energy is applied is indicated by hatching.

By the above-described process, the high-frequency energy is applied at the second output value (steps S2 to S5) to the part PL which is grasped by the low-output areas ArL and the electrodes 912 in the target part TP to extract the extracellular matrix therefrom, so that the part PL is in a close contact state as illustrated in FIG. 8. The heat energy is applied at the second output value to the part PH which is grasped by the high-output area ArH and the first convex portion 911 (step S2 to S5) to extract the extracellular matrix therefrom and the heat energy is applied at the first output value to the part (step S7 to S9) to cauterize the part, so that the part is dissected as illustrated in FIG. 8.

In the medical treatment device 2 according to the first embodiment, the treatment surface 101 includes the high-output area ArH which applies the heat energy to the target part TP at the first output value (the high energy) in which the target part TP is dissected and the low-output area ArL which applies the high-frequency energy to the target part TP at the second output value (the low energy) in which the extracellular matrix of the target part TP is extracted. The high-output area ArH and the low-output area ArL are provided continuously to each other.

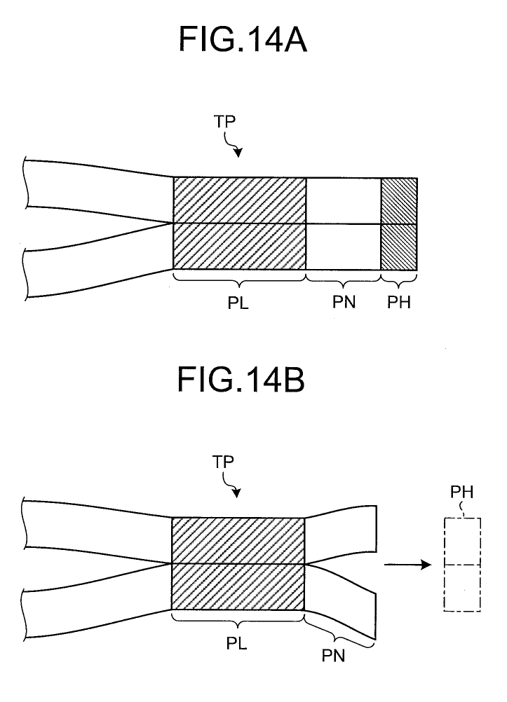

For this reason, in the target part TP subjected to the connecting and dissecting process, the part PN (FIGS. 14A and 14B) which exists between the parts PH and PL in the conventional medical treatment device does not exist as illustrated in FIG. 8.

That is, a time taken until the part PH is separated from the target part TP can be set to a relatively long time compared to the related art. For this reason, since it is possible to prevent the end of the target part TP from being bifurcated in the meantime, the part PL is completely connected by exhibiting the tissue regeneration power.

Thus, according to the medical treatment device 2 of the first embodiment, it is possible to prevent the target part TP from being opened after treatment.

Further, in the medical treatment device 2 according to the first embodiment, the first energy application portion 102 having the high-output area ArH and the second energy application portion 103 having the low-output area ArL are relatively movable from the first positional relation to the second positional relation.

For this reason, when the first and second energy application portions 102 and 103 are set to the second positional relation and the heat energy is applied from the high-output area ArH to the part PH at the first output value while the part PH is grasped at a high pressure, the part PH can be easily dissected.

Further, the medical treatment device 2 according to the first embodiment prompts the operator to perform the second opening and closing operation by turning on the display unit 8 when the impedance of the target part TP reaches the minimum value LV.

For this reason, the part PH can be dissected by the second opening and closing operation of the operator after a sufficient amount of the extracellular matrix is extracted from the target part TP. Thus, the tissue regeneration power of the part PL can be sufficiently exhibited by the extracted extracellular matrix.

Further, the medical treatment device 2 according to the first embodiment applies the heat energy at the first output value from the high-output area ArH to the target part TP after applying the high-frequency energy at the second output value from the low-output area ArL to the target part TP.

For this reason, it is possible to ensure a time necessary until a sufficient amount of the extracellular matrix is extracted from the target part TP. Thus, it is possible to sufficiently exhibit the tissue regeneration power of the part PL by the extracted extracellular matrix.

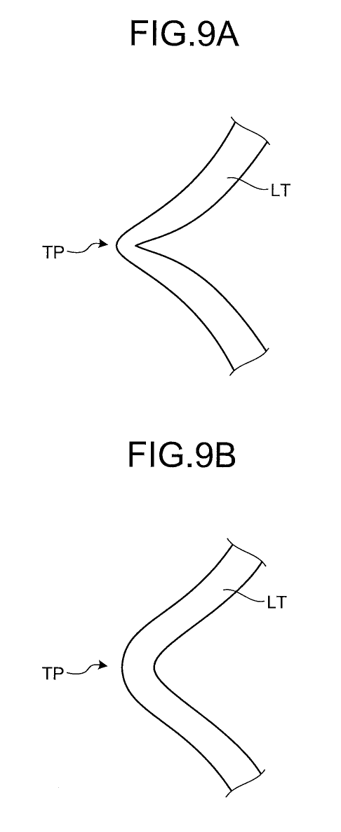

FIGS. 9A and 9B are diagrams illustrating an effect of the first embodiment. Specifically, FIGS. 9A and 9B are diagrams illustrating the body tissue LT after healing by the tissue regeneration power of the part PL. FIG. 9A illustrates a case where the width D1 of the second energy application portion 103 is set to be smaller than the thickness D2 of the target part TP differently from the first embodiment. FIG. 9B illustrates a case where the width D1 is set to be equal to or larger than the thickness D2 as in the first embodiment.

When the width D1 is set to be smaller than the thickness D2, an area of the part PL at the connecting position is relatively small. For this reason, the body tissue LT after healing has a shape in which the target part TP is particular as illustrated in FIG. 9A.

When the width D1 is set to be equal to or larger than the thickness D2 as in the first embodiment, an area of the part PL at the connecting position is sufficiently large. For this reason, as illustrated in FIG. 9B, the body tissue LT after healing does not have a particular shape at the target part TP and is formed so that a boundary line between the target part TP and the other part is smooth.

### Second Embodiment

Next, a second embodiment of the invention will be described.

The same reference signs are used to designate the same elements as those of the first embodiment, and a detailed explanation thereof will be omitted or simplified.

In the medical treatment system according to the second embodiment, the connecting and dissecting process using the control device 3 is different from that of the medical treatment system 1 according to the first embodiment. For this reason, a configuration of the control device and a connecting and dissecting process according to the second embodiment will be described below.

Configuration of Control Device

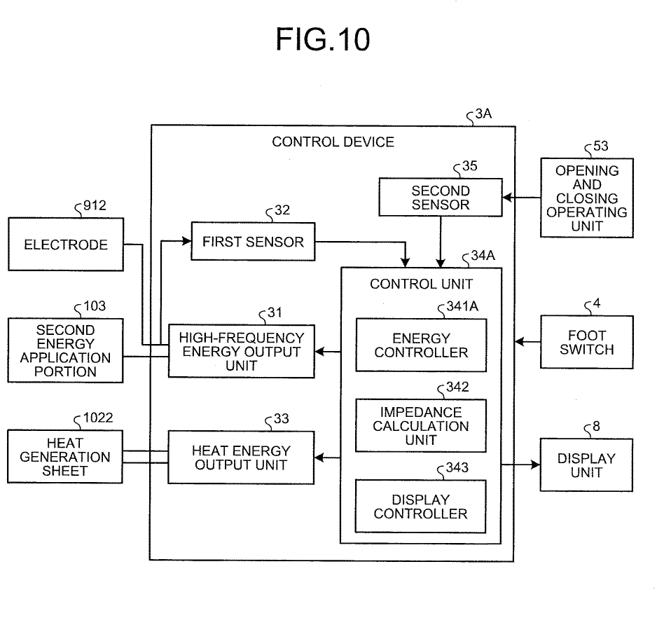

FIG. 10 is a block diagram illustrating a configuration of a control device 3A (overall controller) according to the second embodiment of the invention.

In addition, FIG. 10 mainly illustrates a main part of the invention corresponding to the configuration of the control device 3A.

As illustrated in FIG. 10, the control device 3A according to the second embodiment is different from the control device 3 (FIG. 4) described in the first embodiment in that a second sensor 35 is added and a control unit 34A obtained by changing a part of the function of control unit 34 is employed instead of the control unit 34.

The second sensor 35 detects a movement amount of the cylindrical portion 51 of the opening and closing operating unit 53 in the axial direction (in FIG. 1, the left direction). Then, the second sensor 35 outputs a signal in response to the detected movement amount to the control unit 34A.

As illustrated in FIG. 4, in the control unit 34A, an energy controller 341A obtained by changing a part of the function of the energy controller 341 is employed instead of the energy controller 341 of the control unit 34 (FIG. 4) described in the first embodiment.

A function of the energy controller 341A will be described when the following connecting and dissecting process is described.

Connecting and Dissecting Process

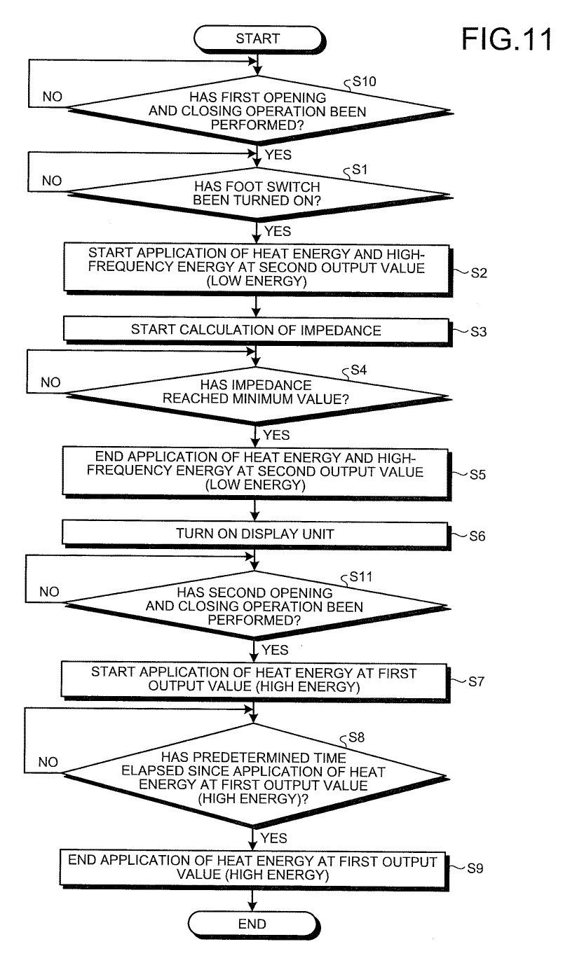

FIG. 11 is a flowchart illustrating the connecting and dissecting process using the control device 3A.

As illustrated in FIG. 11, the connecting and dissecting process according to the second embodiment is different from the connecting and dissecting process (FIG. 5) described in the first embodiment in that steps S10 and S11 are added. For this reason, only steps S10 and S11 will be described below.

Step S10 is performed before step S1.

Specifically, in step S10, the energy controller 341A normally monitors whether the first opening and closing operation is performed by the operator based on the movement amount detected by the second sensor 35. Then, when it is determined that the first opening and closing operation is performed (step S10: Yes), the control device 3A moves the routine to step S1.

That is, the energy controller 341A starts the application of the high-frequency energy and the heat energy at the second output value from the first and second energy application portions 102 and 103 to the target part TP on the condition that the first opening and closing operation is performed (step S10: Yes) and the foot switch 4 is turned on (step S1: Yes) (step S2).

Step S11 is performed after step S6.

Specifically, the energy controller 341A normally monitors whether the second opening and closing operation is performed by the operator based on the movement amount detected by the second sensor 35 in step S11. Then, when it is determined that the second opening and closing operation is performed (step S11: Yes), the control device 3A moves the routine to step S7.

That is, the energy controller 341A starts the application of the heat energy at the first output value from the first energy application portion 102 to the target part TP on the condition that the impedance of the target part TP reaches the minimum value (step S4: Yes) and the second opening and closing operation is performed (step S11: Yes) (step S7).

Even when the connecting and dissecting process which is the same as that of the second embodiment is performed, the same effect as that of the first embodiment is obtained.

### Third Embodiment

Next, a third embodiment of the invention will be described.

The same reference signs are used to designate the same elements as those of the first embodiment, and a detailed explanation thereof will be omitted or simplified.

In the medical treatment system according to the third embodiment, the configuration of the grasping portion 7 is different from that of the medical treatment system 1 of the first embodiment. For this reason, the configuration of the grasping portion according to the third embodiment will be described below.

Configuration of Grasping Portion

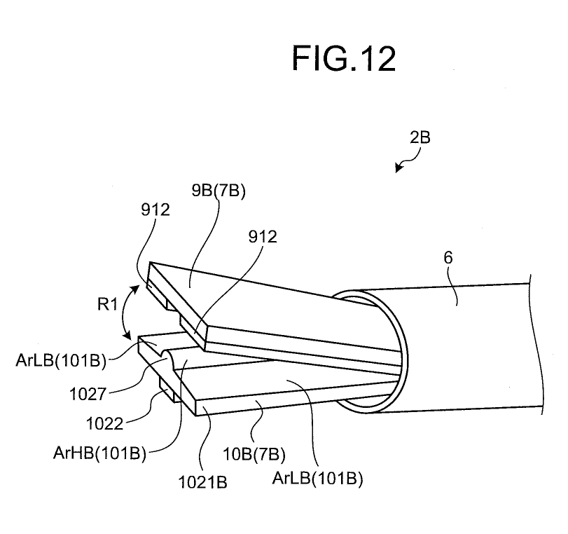

FIG. 12 is an enlarged view illustrating a front end part of a medical treatment device 2B according to the third embodiment of the invention.

As illustrated in FIG. 12, a grasping portion 7B according to the third embodiment includes first and second holding members 9B and 10B which are axially supported by the other end of the shaft 6 and are opened and closed in the direction of the arrow R1 in response to the first and second opening and closing operations of the operator.

The first holding member 9B is a portion which grasps the target part TP between the first holding member 9B and the second holding member 10B and has the same shape as that of the first holding member 9 (including the pair of electrodes 912) described in the first embodiment except that the first convex portion 911 is omitted as illustrated in FIG. 12.

The second holding member 10B is a portion which serves as an energy application portion according to the invention and grasps the target part TP between the first holding member 9B and the second holding member 10B. As illustrated in FIG. 12, the second holding member 10B includes a heat transfer plate 1021B of which only a shape is different from that of the heat transfer plate 1021 described in the first embodiment and a heat generation sheet 1022.

The heat transfer plate 1021B is formed as an elongated plate body similarly to the first holding member 9A and an upper plate surface in FIG. 12 becomes a treatment surface 101B which contacts the target part TP.

As illustrated in FIG. 12, a center portion of the treatment surface 101B in the width direction is provided with a third convex portion 1027 which protrudes upward and extends in the longitudinal direction of the treatment surface 101B.

Here, a cross-section of an outer surface of the third convex portion 1027 has a substantially circular-arc shape.

Both sides of the third convex portion 1027 on the treatment surface 101B (respectively facing the pair of electrodes 912) are downwardly inclined with increasing distance from the third convex portion 1027 in FIG. 12.

As illustrated in FIG. 12, on the treatment surface 101B, an outer surface of the third convex portion 1027 serves as the high-output area ArHB, and each of inclined surfaces on the both sides of the third convex portion 1027 serves as the low-output area ArLB. That is, the high-output area ArHB and the low-output areas ArLB are provided continuously to each other in the width direction of the second holding member 10B. In other words, the high-output area ArHB and the low-output areas ArLB form a continuous surface which is the treatment surface 101B.

More specifically, the both sides of the third convex portion 1027 of the second holding member 10B are electrically connected to the control device 3 via the electric cable C. The both sides of the third convex portion 1027 apply the high-frequency energy to the target part TP when the high-frequency power is supplied from the control device 3 to the pair of electrodes 912 while the low-output areas ArLB contact the target part TP.

As illustrated in FIG. 12, the heat generation sheet 1022 has a width which is the same or slightly smaller than the width of the third convex portion 1027 and the heat transfer plate 1021B adheres to a position facing the third convex portion 1027 in the lower plate surface of the heat transfer plate 1021B in FIG. 12 through an adhesive sheet similarly to the first embodiment. When the heat generation sheet 1022 generates heat due to a voltage applied (energized) from the control device 3 thereto, the heat transfer plate 1021B transfers heat of the heat generation sheet 1022 from the high-output area ArHB to the target part TP (applies the heat energy to the target part TP) while the high-output area ArHB is in contact with the target part TP.

In addition, the connecting and dissecting process according to the third embodiment is the same as the connecting and dissecting process (FIG. 5) described in the first embodiment.

In the following description, a positional relation of the first and second holding members 9B and 10B during the connecting and dissecting process will be described.

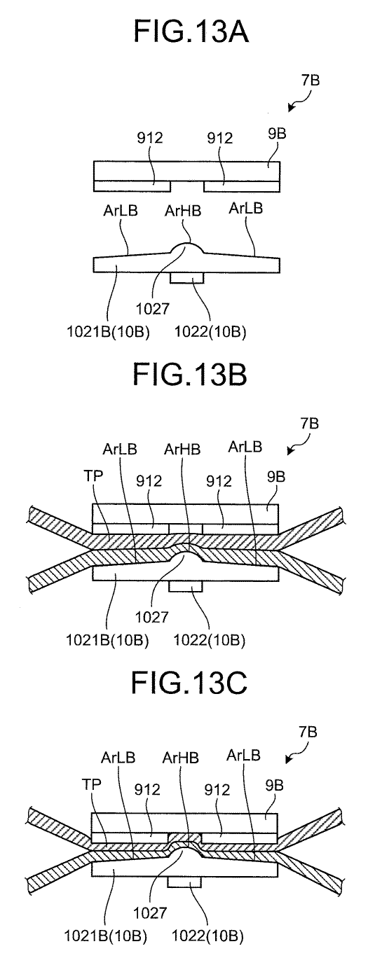

FIGS. 13A to 13C are diagrams illustrating a positional relation of the first and second holding members 9B and 10B during the connecting and dissecting process when viewed from the front end side of the grasping portion 7B. In addition, FIG. 13A illustrates a state where the first and second opening and closing operations are not performed by the operator. FIG. 13B illustrates a state where the first opening and closing operation is performed by the operator. FIG. 13C illustrates a state where the second opening and closing operation is performed by the operator.

When the first opening and closing operation is performed by the operator, the first and second holding members 9B and 10B move close to each other to grasp the target part TP as illustrated in FIGS. 13A and 13B. The state illustrated in FIG. 13B is continued during steps S1 to S6.

Then, when the second opening and closing operation is performed by the operator in response to the on state of the display unit 8 in step S6, the first and second holding members 9B and 10B further move close to each other as illustrated in FIGS. 13B and 13C. That is, in the target part TP, a part which is grasped by the third convex portion 1027 and the first holding member 9B is grasped at a pressure higher than those of the other parts. The state illustrated in FIG. 13C is continued during steps S7 to S9.

Even if the grasping portion 7B of the third embodiment is employed, the same effect as that of the first embodiment is obtained.

Further, the structure of the grasping portion 7B can be simplified if the grasping portion 7B of the third embodiment is employed.

### Other Embodiments

Embodiments for carrying out the invention have been described so far, but the invention is not limited to the first to third embodiments.

In the first to third embodiments, the heat energy is applied from the high-output area ArH (ArHB) to the target part TP and the high-frequency energy is applied from the low-output area ArL (ArLB) to the target part TP, but the invention is not limited thereto. If at least one of the heat energy, the high-frequency energy, and the ultrasonic energy can be applied, two different types of energy may be applied as in the first to third embodiments or only one type of energy may be applied.

In the first to third embodiments, the energy application portion according to the invention is provided only in the second holding member 10 (10B), but the invention is not limited thereto. The energy application portion may be provided in at least one of the first and second holding members 9 and 10 (9B and 10B) or both the first and second holding members 9 and 10 (9B and 10B).

In the first to third embodiments, two low-output areas ArL (ArLB) are provided, but the invention is not limited thereto. For example, only one low-output area may be provided.

In the first to third embodiments, a process from step S5 is performed based on the impedance of the target part TP, but the invention is not limited thereto. For example, a process from step S5 may be performed based on physical property values relating to the hardness, thickness, or temperature of the target part TP.

In the first to third embodiments, a process from step S5 is performed after the impedance of the target part TP reaches the minimum value VL, but the invention is not limited thereto. If the time is after the time T1 at which the impedance of the target part TP reaches the minimum value VL (for example, a time from the time T1 to the time T2 (FIG. 7) which returns to the initial value VI (FIG. 7) at a time point at which the application of the heat energy and the high-frequency energy at the second output value starts), a process from step S5 may be performed at any timing. In order to simplify a control, a process from step S5 may start at a timing when a certain period of time has elapsed since the start of the application of the energy to the target part TP without particularly monitoring the impedance.

In the first to third embodiments, the first and second holding members 9 and 10(9B, 10B) or the pressing member 11 is opened and closed (manually opened and closed) in response to the first and second opening and closing operations of the operator, but the invention is not limited thereto. For example, a motor or the like may be provided in the medical treatment device 2 (2B) and the first and second holding members 9 and 10 (9B and 10B) or the pressing member 11 is opened and closed at an appropriate timing under the control of the control unit 34 (34A).

In the first to third embodiments, the second opening and closing operation is requested in such a manner that the display unit 8 configured as an LED and serving as the output device according to the invention is turned on, but the invention is not limited thereto. For example, a message may be displayed or sound may be generated so as to request the second opening and closing operation.

In the medical treatment system including the grasping portion 7B according to the third embodiment, the connecting and dissecting process described in the second embodiment may be performed.

The flow of the connecting and dissecting process is not limited to the procedure of the connecting and dissecting process (FIG. 5, FIG. 11) described in the first to third embodiments and may be modified within a range without inconsistency.

According to the medical treatment device of some embodiments, it is possible to prevent a target part from being opened after treatment.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical treatment device comprising a pair of holding members configured to grasp a target part to be connected and dissected in a body tissue,
at least one holding member of the pair of holding members comprising an energy application portion having a treatment surface configured to contact the target part when the target part is grasped by the pair of holding members to apply energy to the target part,
the treatment surface comprising:
a high-output area configured to apply energy to the target part at a first output value for dissecting at least the target part; and
a low-output area configured to apply energy to the target part at a second output value smaller than the first output value, and
wherein the high-output area and the low-output area are provided continuously to each other.

2. The medical treatment device according to claim 1,
wherein the second output value is an output value for extracting at least an extracellular matrix of the target part.

3. The medical treatment device according to claim 1,
wherein the energy application portion comprises:
a first energy application portion having the high-output area; and
a second energy application portion having the low-output area, and
wherein the first energy application portion and the second energy application portion are relatively movable between a first positional relation in which the high-output area and the low-output area are located at a same height and a second positional relation in which the high-output area protrudes relative to the low-output area.

4. The medical treatment device according to claim 3, further comprising:
an operating device provided for user operation to change the first energy application portion and the second energy application portion between the first positional relation and the second positional relation;
an overall controller configured to calculate an impedance of the target part; and
an output device configured to inform information prompting the user operation after the impedance of the target part calculated by the overall controller reaches a minimum value.

5. The medical treatment device according to claim 1,
wherein the energy application portion has such a shape that the high-output area and the low-output area are integrated together, and the high-output area protrudes relative to the low-output area.

6. The medical treatment device according to claim 1, further comprising an overall controller configured to be electrically connected to the energy application portion and cause the energy application portion to apply energy to the target part,
wherein the overall controller is configured to cause the energy application portion to apply energy from the high-output area to the target part at the first output value after applying energy from the low-output area to the target part at the second output value.

7. The medical treatment device according to claim 6,
wherein the overall controller is configured to calculate an impedance of the target part, and cause the energy application portion to apply energy from the high-output area to the target part at the first output value after the impedance of the target part reaches a minimum value.

8. The medical treatment device according to claim 6,
wherein the energy application portion comprises:
a first energy application portion having the high-output area; and
a second energy application portion having the low-output area,
wherein the first energy application portion and the second energy application portion are relatively movable between a first positional relation in which the high-output area and the low-output area are located at a same height and a second positional relation in which the high-output area protrudes relative to the low-output area, and
wherein the overall controller is configured to cause the energy application portion to:
apply energy from the low-output area to the body tissue at the second output value when the high-output area and the low-output area have the first positional relation; and
apply energy from the high-output area to the body tissue at the first output value when the high-output area and the low-output area have the second positional relation.
